(19) United States
(12) Patent Application Publication
(10) Pub. No.: US 2005/0257328 A1
(43) Pub. Date: Nov. 24, 2005

(54) Agent for simultaneously dying and bringhtening keratin fibers

(76) Inventors: Anette Sallwey, Dreieich (DE); Hayel Said, Simi Valley, CA (US)

Correspondence Address: Striker Striker & Stenby, 103 East Neck Road, Huntington, NY, 11743, US

(21) Appl. No.: 10/528,985

(22) PCT Filed: Nov. 21, 2003
(86) PCT No.: PCT/EP03/13066

§ 371 Date: Mar. 24, 2005

(30) Foreign Application Priority Data
Mar. 6, 2003 (DE) 20303559.3

(51) Int. Cl. A61K 7/13

(52) U.S. Cl. 8/405

(57) ABSTRACT

The present patent application concerns an agent for the simultaneous brightening and coloring of keratin fibers, said agent being characterized in that it contains at least one peroxy salt and at least one dye resistant to peroxy salts which is taken from the group consisting of azo dyes, quinone dyes, triphenylmethane dyes, nitro dyes, acid dyes and basic dyes.

The object of the present patent application is an agent for simultaneous coloring and brightening of keratin fibers, for example wool, silk or hair and particularly human hair.

Brightening and coloring formulations with direct and oxidative dyes are known. As a result of the ammonia they contain and of the addition of a peroxide, these formulations bring about a brightening of 1 to 2 shade gradations. On previously dyed hair in particular, however, the brightening with these formulations is only very slight. This low brightening efficacy greatly limits the number of achievable shades.

The objective was therefore to provide a brightening coloring agent that would permit a brightening of up to six shade gradations and a broad spectrum of shades. In particular, the brightening and coloring agent was to be applicable to both natural hair and previously dyed hair.

We have now found that the aforesaid objective can be reached in outstanding manner by use of a combination of certain direct dyes with peroxy salts that afford colorations with unusually good color intensity, covering power and durability.

The object of the present invention therefore is an agent for the simultaneous brightening and coloring of keratin fibers, said agent containing peroxy salts and peroxy salts-resistant dyes selected from the group consisting of azo dyes, quinone dyes, triphenylmethane dyes, acid dyes, basic dyes and nitro dyes.

The total amount of the afore-said direct dyes in the brightening colorant is from about 0.001 to 10 weight percent and particularly from 0.002 to 8 weight percent. The concentration of these direct dyes in the ready-to-use brightening agent that has been mixed with hydrogen peroxide is from about 0.0005 to 5 weight percent.

Useful direct dyes for the attainment of the desired color shades are common, physiologically harmless dyes resistant to peroxy salts and selected from the group consisting of azo dyes, quinone dyes, triphenylmethane dyes, acid dyes, basic dyes and nitro dyes or a combination thereof, for example: 3-(2′,6′-diaminopyridyl-3′-azo)pyridine {=2,6-diamino-3-[(pyridin-3-yl)azo]pyridine}; 2-[(4-ethyl-(2-hydroxyethyl)amino]-2-methylphenyl)-azo]-5-nitro-1,3-thiazole (Disperse Blue 106); N,N-di(2-hydroxyethyl)-3-methyl4-[(4-nitro-phenyl)azo]aniline (Disperse Red 17, CI 11210); 3-diethylamino-7-(4-dimethylamino-phenylazo)-5-phenylphenazinium chloride (CI 11050); 4-(2-thiazolylazo)resorcinol; sodium 4-[(4-phenylamino)azo]benzenesulfonate (Orange IV); 1 -[(3-aminopropyl)amino-9,10-an-thracenedione (HC Red No. 8); 3′,3″,4,5,5′,5″,6,7-octabromophenolsulfonephthalein (Tetrabromophenol Blue); 1-[(4-amino-3,5-dimethylphenyl)-(2,6-dichlorophenyl)methylene]-3,5-dimethyl-4-imino-2,5-cyclohexadiene phosphoric acid (1:1) (Basic Blue 77); 3′,3″,5′,5″-tetrabromo-m-cresolsulfonephthalein; disodium 2,4-dinitro-1-naphthol-7-sulfonate (Acid Yellow 1; CI 10 316); sodium 4-[(2′-hydroxy-1′-naphthyl)azo]benzenesulfonate (Acid Orange 7; CI 15 510); 3′,6′-dihydroxy-2′,4′,5′,7′-tetraiodospiro[isobenzofuran-1(3H),9′(9H)-xanthene]-3-one disodium salt (Acid Red 51; CI 45 430); disodium 6-hydroxy-5-[(2-methoxy-5-methyl-4-sulfophenyl)azo]-2-naphthalenesulfonate (FD&C Red 40; CI 16035); 2,4-dinitro-1-naphthol sodium salt (Acid Yellow 24; CI 10315); 2′,4′,5′,7′-tetrabromo-4,5,6,7-tetrachloro-3′,6′-dihydroxyspiro[isobenzofuran-1(3H),9′(9H)xanthene]-3-one disodium salt (Acid Red 92; CI 45410); sodium 4-(2-hydroxy-1-naphthylazo)-3-methyl-benzenesulfonate (Acid Orange 8; CI 15575); 2-amino-1,4-naphtalenedione; dithizone (1,5-diphenyl-thiocarbazone); N-(2-hydroxyethyl)-2-nitro-4-(trifluoromethyl)aniline (HC Yellow 13); N-(2-hydroxyethyl)-4-nitroaniline and 4-chloro-N-(2,3-dihydroxypropyl)-2-nitroaniline.

The amount of dyes, based on the total amount of the brightening colorant, is from about 0.001 to 10 weight percent and particularly from 0.002 to 8 weight percent.

Preferably used peroxy salts are the persulfates, for example, sodium persulfate, potassium per-sulfate, ammonium persulfate or mixtures of these persulfates.

The concentration of the peroxy salts in the brightening colorant is from about 0.1 to 50 weight percent and particularly from 1 to 40 weight percent.

Other constituents of the brightening colorant of the invention are, in general, surfactants and emulsifiers from the group of anionic, nonionic or ampholytic surface-active compounds, for example fatty alcohol sulfates, alkane sulfonates, olefin sulfonates, fatty alcohol polyglycol ether sulfates, alkyl polyglycosides and ethoxylated fatty alcohols, fatty acids, alkylphenols, sorbitan fatty esters and fatty alkanolamides; thickeners and gel formers, for example fatty alcohols, fatty acids, paraffin oils, fatty esters, methylcellulose, or hydroxyethylcellulose, starch, synthetic polymers such as polyvinypyrrolidone and polyacrylates, or biopolymers such as alginic acid; stabilizers for peroxo compounds, for example silicates; as well as complexing agents; perfume oils and hair-care additives such as cationic polymers, lanolin derivatives, cholesterol, pantothenic acid, protein derivatives and protein hydrolyzates, provitamins and vitamins as well as plant extracts, alkali metal sulfates or alkaline earth metal sulfates, for example sodium sulfate and ammonium sulfate, alkali metal stearates or alkaline earth metal stearates, for example sodium stearate, and aluminum stearate. In the preparation of the brightening colorant of the invention, these additives are used in amounts commonly employed for such purposes; for example the surfactants and emulsifiers at a concentration from about 0.2 to 30 weight percent and the thickeners at a concentration from about 0.1 to 30 weight percent (always based on the ready-to-use brightening colorant).

The pH of the ready-to-use brightening colorant is usually from about 6 to 12, a pH from about 6.5 to 10.5 and particularly from 7 to 10 being preferred.

The pH is preferably adjusted with alkali metal or alkaline earth metal salts that are alkaline in aqueous solution, for example sodium carbonate, sodium hydrogen carbonate, magnesium carbonate, ammonium carbonate, ammonium hydrogen carbonate, sodium silicate or mixtures of these salts. The concentration of these salts, based on the total amount of the brightening colorant, is from 0.1 to 60 weight percent and particularly from 0.5 to 55 weight percent.

The brightening colorant of the invention is formulated, in admixture with the other components usually contained in bleaching agents, as a cosmetic composition in the form of a water-free powder or in a water-free, liquid or creamy medium (“coloring paste”). Powder formulations are usually dedusted by spraying with inert carrier materials, for example paraffin oils, silicone oils, polyethers, fatty esters, polyorganosiloxanes [for example with dimethylpolysiloxanes referred to in the International Cosmetic Ingredient Dictionary, 5th ed., pp. 220/221 (1993) under the name “dimethicone”] or waxes. Such dedusting methods are known, for example, from U.S. Pat. No. 5,698,186 and U.S. Pat. No. 5,891,423.

Depending on the direct dye used, the dyes can also be in the form of an aqueous solution which is stored separately from the water-free peroxy salt-containing composition and which is mixed with the brightening agent only just before use. The colorant can also be in a microencapsulated form or packaged in a water-soluble covering (for example in a polyvinyl alcohol or polyvinylpyrrolidone pouch) and released only upon mixing with the peroxide composition.

To use the brightening colorant of the invention, the powder is mixed before use with an aqueous hydrogen peroxide preparation to form an applicable coloring paste, the mixing possibly being carried out in a bowl or by shaking in an application bottle. The hydrogen peroxide preparation used for this purpose (for example an aqueous solution or emulsion) contains about 0.5 to 20 weight percent of hydrogen peroxide or of an addition salt thereof, an amount of 1.5 to 12 weight percent being preferred.

The mixing ratio of the colorant of the invention to the hydrogen peroxide preparation is about 1:1 to 1:3 and preferably 1:1.5 to 1:2.

The ready-to-use colorant thus obtained (“coloring paste”) is applied uniformly to dry fibers (particularly human hair). After an exposure time from about 5 to 80 minutes at 20 to 50° C., preferably 15 to 60 minutes at room temperature (20 to 25° C.), or 10 to 50 minutes by supplying heat (30 to 50° C.), this is followed by rinsing with water and drying.

The colorant of the invention is preferably packaged together with the hydrogen peroxide preparation in the form of a 2-component kit. It is, however, entirely possible to have the colorant and the hydrogen peroxide preparation in two separate packages.

By means of the colorant of the invention, keratin fibers can be brightened and colored in a single step, it being possible to color previously dyed hair after a very short time. The colorations achieved with the colorant of the invention show very high color intensity, covering power and durability.

The following examples will explain the subject matter in greater detail without limiting its scope.

### EXAMPLES

### Example 1

### Brightening Colorant

| | |
|---|---|
| Potassium persulfate | 20.0 |
| Ammonium persulfate | 30.0 |
| Sodium silicate | 24.0 |
| Magnesium oxide | 12.5 |
| Hydroxyethyl cellulose | 5.0 |
| Soap beads | 6.0 |
| Disperse silicic acid | 2.0 |
| Disodium EDTA | 0.5 |

The dyes were worked into the above-indicated powder base. The resulting coloring powder was then mixed with 12% aqueous peroxide solution in a 1:2 ratio, stirred in a bowl until homogeneous and then applied to dry, dark-blond natural hair. After an exposure time of 20 minutes (Table 1) or 45 minutes (Table 2) at 40° C., the hair was rinsed with warm water and dried. The dyes used and the coloring result obtained are indicated in Tables 1 and 2.

TABLE 1
Exposure time: 20 minutes at 40° C.

| | | Coloring Result |
|---|---|---|
| | | (a) 0.5% of dye |
| | | (b) 2% of dye |
| Dye used | CI No. | in the brightening powder base |
| Acid Yellow 1 | 10316 | (a) yellow |
| | | (b) lemon yellow |
| FD&C Red 40 | 16035 | (a) delicate pink |
| | | (b) delicate pink |
| Acid Yellow 24 | 10315 | (a) intense brilliant lemon yellow |
| | | (b) intense brilliant lemon yellow |
| Acid Red 92 | 45410 | (a) intense brilliant pink |
| | | (b) intense brilliant pink |
| Tetrabromophenol Blue | — | (a) intense cobalt blue |
| | | (b) intense cobalt blue |
| 3-(2′,6′-Diaminopyridyl- | — | (a) weak warm yellow |
| 3′-azo)pyridine | | (b) warm yellow |
| Acid Orange 7 | 15510 | (a) weak orange |
| | | (b) intense brilliant orange |
| Disperse Blue 106 | — | (a) weak gray-green-blue |
| | | (b) weak gray-green-blue |
| Basic Blue 77 | — | (a) weak light blue |
| | | (b) light blue |
| Acid Orange 8 | 15575 | (a) red orange |
| | | (b) red orange |
| Disperse Red 17 | 11210 | (a) dusky pink |
| | | (b) bluish red |
| Acid Red 51 | 45430 | (a) brilliant pink |
| | | (b) brilliant pink |
| 3′,3″,5′,5″-Tetra- | — | (a) green blue |
| bromo-m- | | (b) dark green blue |
| cresolsulfonephthalein | | |
| 3-Diethylamino-7- | 11050 | (a) gray brown |
| (4-dimethylamino- | | (b) gray brown |
| phenylazo)-5-phenyl- | | |
| phenazinium chloride | | |
| 4-(2-Thiazolylazo)- | — | (a) red orange |
| resorcinol | | (b) red orange |
| Dithizone | — | (a) red orange |
| | | (b) red orange |
| HC Red No. 8 | — | (a) delicate bluish pink |
| | | (b) bluish pink |
| 2-Amino-1,4- | — | (a) orange yellow |
| naphthalenedione | | (b) orange yellow |
| HC Yellow 13 | — | (a) intense brilliant lemon yellow |
| | | (b) intense brilliant lemon yellow |
| Orange IV | 13080 | (a) orange yellow |
| | | (b) orange yellow |
| N-(2-Hydroxyethyl)- | — | (a) intense brilliant lemon yellow |
| 4-nitroaniline | | (b) intense brilliant lemon yellow |
| 4-Chloro-N-(2,3- | — | (a) orange yellow |
| dihydroxypropyl)- | | (b) orange yellow |
| 2-nitroaniline | | |

TABLE 2
Exposure time 45 minutes at 40° C.

| | | Coloring Result |
|---|---|---|
| | | (a) 0.5% of dye |
| | | (b) 2% of dye |
| Dye used | CI No. | in the brightening powder base |
| Acid Yellow 1 | 10316 | (a) yellow |
| | | (b) lemon yellow |
| FD&C Red 40 | 16035 | (a) delicate pink |
| | | (b) bluish red |
| Acid Yellow 24 | 10315 | a) intense brilliant lemon yellow |
| | | (b) intense brilliant lemon yellow |
| Acid Red 92 | 45410 | (a) intense brilliant pink |
| | | (b) intense brilliant pink |
| Tetrabromophenol Blue | — | (a) intense cobalt blue |
| | | (b) intense cobalt blue |
| 3-(2′,6′-Diaminopyridyl- | — | (a) warm yellow |
| 3′-azo)pyridine | | (b) warm dark yellow |
| Acid Orange 7 | 15510 | (a) orange |
| | | (b) intense brilliant orange |
| Disperse Blue 106 | — | (a) weak gray-green-blue |
| | | (b) weak gray-green-blue |
| Basic Blue 77 | — | (a) weak light blue |
| | | (b) light blue |
| Acid Orange 8 | 15575 | (a) red orange |
| | | (b) red orange |
| Disperse Red 17 | 11210 | (a) dusky pink |
| | | (b) bluish red |
| Acid Red 51 | 45430 | (a) brilliant pink |
| | | (b) brilliant pink |
| 3′,3″,5′,5″-Tetra- | — | (a) green blue |
| bromo-m- | | (b) dark green blue |
| cresolsulfonephthalein | | |
| 3-Diethylamino-7- | 11050 | (a) light gray |
| (4-dimethylamino- | | (b) light gray |
| phenylazo)-5-phenyl- | | |
| phenazinium chloride | | |
| 4-(2-Thiazolylazo)- | — | (a) red orange |
| resorcinol | | (b) red orange |
| Dithizone | — | (a) red orange |
| | | (b) red orange |
| HC Red No. 8 | — | (a) delicate bluish pink |
| | | (b) bluish pink |
| 2-Amino-1,4- | — | (a) orange yellow |
| naphthalenedione | | (b) orange yellow |
| HC Yellow 13 | — | (a) intense brilliant lemon yellow |
| | | (b) intense brilliant lemon yellow |
| Orange IV | 13080 | (a) orange yellow |
| | | (b) orange yellow |
| N-(2-Hydroxyethyl)- | — | (a) intense brilliant lemon yellow |
| 4-nitroaniline | | (b) intense brilliant lemon yellow |
| 4-Chloro-N-(2,3- | — | (a) orange yellow |
| dihydroxypropyl)- | | (b) orange yellow |
| 2-nitroaniline | | |

### Example 2

### Brightening Colorant

| | |
|---|---|
| Potassium persulfate | 20.0 |
| Ammonium persulfate | 30.0 |
| Sodium silicate | 24.0 |
| Magnesium oxide | 12.5 |
| Hydroxyethylcellulose | 5.0 |
| Soap beads | 6.0 |
| Disperse silicic acid | 2.0 |
| Disodium EDTA | 0.5 |

The dyes indicated in Table 3 together with 0.5% of Acid Red 92 were worked into the above-described brightening powder base. The resulting coloring powder was then mixed with a 12% aqueous peroxide solution in a 1:2 ratio and stirred in a mixing bowl until homogeneous, after which it was applied to dry dark-brown natural hair. After an exposure time of 20 minutes at 40° C., the strands were rinsed with warm water and dried.

TABLE 3

| Dye used | | | |
|---|---|---|---|
| (Amount in %) | | CI No. | Coloring Result |
| Acid Yellow 1 | (0.5) | 10316 | intense brilliant pink |
| Acid Yellow 24 | (0.5) | 10315 | red |
| Acid Yellow 24 | (2.0) | 10315 | red |
| Tetrabromophenol | (0.5) | — | violet |
| Blue | | | |
| Tetrabromophenol | (2.0) | — | intense warm blue |
| Blue | | | |
| Acid Orange 7 | (0.5) | 15510 | intense brilliant pink |
| Acid Orange 7 | (2.0) | 15510 | brilliant red |
| Disperse Blue 106 | (0.5) | — | bluish red |
| Disperse Blue 106 | (2.0) | — | bluish red |
| Basic Blue 77 | (0.5) | — | bluish red |
| Basic Blue 77 | (2.0) | — | bluish red |
| Acid Orange 8 | (0.5) | 15575 | intense brilliant pink |
| Acid Orange 8 | (2.0) | 15575 | intense brilliant pink |
| Disperse Red 17 | (0.5) | 11210 | intense brilliant pink |
| Disperse Red 17 | (2.0) | 11210 | intense pink |

### Example 3

### Brightening Colorant

| | |
|---|---|
| Potassium persulfate | 20.0 |
| Ammonium persulfate | 30.0 |
| Sodium silicate | 24.0 |
| Magnesium oxide | 12.5 |
| Hydroxyethylcellulose | 5.0 |
| Soap beads | 6.0 |
| Disperse silicic acid | 2.0 |
| Disodium EDTA | 0.5 |
| Acid Red 92 (CI 45410) | 0.01 |
| Tetrabromophenol Blue | 0.01 |
| Basic Blue 77 | 0.10 |

The above coloring powder was mixed with a 12% aqueous peroxide solution in a 1:2 ratio in a mixing bowl until homogeneous and then applied to dry, oxidatively dyed light-brown hair. After an exposure time of 20 minutes at 40° C., the strands were rinsed with warm water and dried.

The hair was colored to a delicate bluish pink shade.

### Example 4

### Brightening Colorant

| | |
|---|---|
| Potassium persulfate | 20.0 |
| Ammonium persulfate | 30.0 |
| Sodium silicate | 24.0 |
| Magnesium oxide | 12.5 |
| Hydroxyethylcellulose | 5.0 |
| Soap beads | 6.0 |
| Disperse silicic acid | 2.0 |
| Disodium EDTA | 0.5 |
| Acid Red 92 (CI 45410) | 0.1 |
| Acid Yellow 1 (CI 10316) | 0.2 |
| Acid Yellow 24 (CI 10315) | 0.8 |
| Acid Orange No. 8 (CI 15575) | 1.0 |
| Acid Orange No. 7 (CI 15510) | 1.0 |

The above coloring powder was mixed with a 12% aqueous peroxide solution in a 1:2 ratio in a mixing bowl until homogeneous and then applied to dry, oxidatively dyed black hair. After an exposure time of 20 minutes at 40° C., the strands were rinsed with warm water and dried.

The hair was colored to a brilliant orange shade.

### Example 6

### [sic] Brightening Colorant

Brightening Coloring Powder

| | |
|---|---|
| Potassium persulfate | 20.00 |
| Ammonium persulfate | 30.00 |
| Sodium silicate | 24.00 |
| Magnesium oxide | 12.50 |
| Hydroxyethylcellulose | 5.00 |
| Soap beads | 6.00 |
| Disperse silicic acid | 2.00 |
| Disodium EDTA | 0.50 |
| Acid Red 92 (CI 45410) | 0.10 |
| Acid Yellow 1 (CI 10316) | 0.15 |
| Acid Yellow 24 (CI 10315) | 0.05 |
| Acid Orange No. 8 (CI 15575) | 0.02 |
| Acid Orange No. 7 (CI 15510) | 0.20 |
| 3-(2′,6′-Diaminopyridyl-3′-azo)- | 0.10 |
| pyridine | |
| Acid Red 51 (CI 45430) | 0.20 |
| 3′,3″,5′,5″-Tetrabromo-m-cresol- | 0.20 |
| sulfonephthalein | |
| Tetrabromophenol Blue | 0.05 |
| Disperse Blue 106 | 0.10 |
| Basic Blue 77 | 0.10 |
| Disperse Red 17 (CI 11210) | 0.01 |

The above coloring powder was mixed with a 12% aqueous peroxide solution in a 1:2 ratio in a mixing bowl until homogeneous and then applied to dry, oxidatively dyed black hair. After an exposure time of 20 minutes at 40° C., the strands were rinsed with warm water and dried.

The hair was colored to a gray brown shade.

Unless otherwise indicated, all percentages given in the present patent application are by weight.

1. Agent for the simultaneous brightening and coloring of keratin fibers, characterized in that it contains at least one peroxy salt and at least one dye resistant to peroxy salts which is taken from the group consisting of azo dyes, quinone dyes, triphenylmethane dyes, acid dyes and basic dyes [sic].

2. Agent according to claim 1, characterized in that the dye resistant to peroxy salts is selected from among 3(2′,6′-diaminopyridyl-3′-azo)pyridine; 2-[(4-ethyl-(2-hydroxyethyl)-amino]-2-methylphenyl)azo]}-nitro-1,3-thiazole; N N-di(2-hydroxyethyl)-3-methyl-4-[(4-nitrophenyl)azo]aniline; 3-diethylamino-7-(4-dimethylaminophenylazo)-5-phenylphenazinium chloride; 4-(2-thiazolylazo)resorcinol; sodium 4-[(4-phenylamino)azo]benzenesulfonate; 1-[(3aminopropyl)amino-9,10-anthracenedione; 3′,3″,4,5,5′,5″,6,7-abromophenolsulfone-phthalein; 1-[(4-amino-3,5-dimethylphenyl)-(2,6-dichlorophenyl)methylene]-3,5-dimethyl-4-imino-2,5-cyclohexadiene phosphoric acid (1:1) (Basic Blue 77); 3′,3″,5′,5″-tetrabromo-m-cresolsulfonephthalein; disodium 2,4-dinitro-1-naphthol-7-sulfonate; sodium 4-2′-hydroxy-1′-naphthyl)-azo]benzenesufonate; 3′,6′-dihydroxy-2′,4′,5′,7′-tetraiodospiro-[(isobenzofuran-1(3H)-9′(9H)-xanthen]-3-one disodium salt; disodium 6-hydroxy-5-[(2-methoxy-5-methyl-4-sulfophenyl)azo]-2-naphthalenesulfonate; 2,4-dinitro-1-naphthol sodium salt; 2′,4′,5′,7′-tetrabromo-4,5,6,7-tetrachloro-3′,6′-dihydroxyspiro[isobenzofuran-1(3H), 9′(9H)xanthene]-3-one disodium salt; sodium 4-(2-hydroxy-1-naphthylazo)-3-methylbenzenesulfonate; 2-amino-1,4-naphtalenedione; dithizone; N-(2-hydroxyethyl)-2-nitro-4-(trifluoromethyl)aniline; N-(2-hydroxyethyl)-4-nitroaniline and 4-chloro-N-(2,3-dihydroxypropyl)-2-nitroaniline.

3. Agent according to claim 1, characterized in that it contains the dye in an amount from 0.001 to 10 weight percent.

4. Agent according to claim 1, characterized in that the peroxy salt is selected from among sodium persulfate, potassium persulfate, ammonium persulfate or mixtures of said persulfates.

5. Agent according to claim 1, characterized in that it contains the peroxy salt in a total amount from 0.1 to 50 weight percent.

6. Agent according to claim 1, characterized in that in the ready-to-use condition it has a pH of 6 to 12.

7. Two-component kit consisting of a colorant and a hydrogen peroxide preparation, characterized in that the colorant is an agent according to claim 1.

8. Agent according to claim 7, characterized in that the hydrogen peroxide preparation is an aqueous solution or emulsion.

9. Agent according to claim 7, characterized in that the hydrogen peroxide preparation contains from 0.5 to 20 weight percent of hydrogen peroxide or an addition salt thereof.
